(19) United States
(12) Patent Application Publication
(10) Pub. No.: US 2025/0243135 A1
(43) Pub. Date: Jul. 31, 2025

(54) REDOX CATALYSTS AND PROCESSES FOR EFFICIENT OXIDATIVE DEHYDROGENATION OF ALKYLAROMATICS

(71) Applicant: North Carolina State University, Raleigh, NC (US)

(72) Inventors: Fanxing Li, Cary, NC (US); Junchen Liu, Kingsport, TN (US)

(21) Appl. No.: 18/871,198

(22) PCT Filed: Jun. 1, 2023
(86) PCT No.: PCT/US2023/024115

§ 371 Date: Dec. 3, 2024

Related U.S. Application Data
(60) Provisional application No. 63/348,550, filed on Jun. 3, 2022.

(51) Int. Cl. C07C 5/32; B01J 23/00; B01J 23/34; B01J 23/78; B01J 23/83; B01J 23/889; B01J 37/03; B01J 37/08; B01J 37/18; B01J 38/12

(52) U.S. Cl. CPC C07C 5/322; B01J 23/002; B01J 23/34; B01J 23/78; B01J 23/83; B01J 23/8892; B01J 37/036; B01J 37/086; B01J 37/18; B01J 38/12; B01J 2523/13; B01J 2523/23; B01J 2523/3706; B01J 2523/3718; B01J 2523/72; B01J 2523/842

(57) ABSTRACT

The present invention provides novel redox catalysts and processes using the redox catalyst for the oxidative dehydrogenation of alkylaromatics. The catalyst enables the dehydrogenation reaction to produce an alkenylaromatic and hydrogen, the selective hydrogen combustion reaction of lattice oxygen with the hydrogen to produce steam, and carbon monoxide/dioxide (COₓ) management by mitigating COₓ production, capturing the COx, and/or or otherwise providing COₓ resistance.

### CROSS-REFERENCE

This application is claims priority to PCT Application No. PCT/US23/24115 filed on Jun. 1, 2023, which claims priority to U.S. Application No. 63/348,550, filed on Jun. 3, 2022, both of which are incorporated herein by reference in their entirety.

### FIELD OF THE INVENTION

The present invention relates generally to redox catalysts. The invention pertains to a redox catalyst comprising a catalytic dehydrogenation (DH) component, a COₓ management (mitigation and resistance) component, and an optional enhanced SHC function within the COₓ management component or an optional SHC component. The redox catalyst comprises one of the optional enhanced SHC function or the optional SHC component.

### BACKGROUND

As important chemical building blocks, alkenylaromatics are often used as precursors to important polymers, synthetic lubricants, hydroperoxides, protective coatings and personal care ingredients such as polystyrene and poly-α-methyl styrene resin. State-of-the-Art (SoTA) production of alkenylaromatics is thermal catalytic dehydrogenation, with dilution of steam (steam cracking). For example, 90% of the styrene is industrially produced via steam cracking of ethylbenzene. Current estimation indicates that by the year of 2028, the global styrene market would be ˜$71 billion, representing a 34% predicted growth from 2022. However, current practice leads to several implications regarding energy consumption of CO₂ emission: 1) the reaction equilibrium limitation of the current steam cracking technology limited single-pass yield of the ethylbenzene to ˜60%. 2) The endothermic nature of the reaction requires constant energy inputs. 3) Significant steam dilution is required for catalytic dehydrogenation; 4) Co-produced H₂ requires complex separation and incur high energy consumption.

Industrially available technologies such as BASF StyroStar®, ABB Lummus/UOP, and Fina/Badger technologies involve cofeeding a large amount of steam (volumetric S/O≥10:1) as heat carrier and reaction atmosphere in the catalytic dehydrogenation (DH) process. Oxidative dehydrogenation (ODH) approach has been demonstrated by co-feeding gaseous oxygen to selectively combust hydrogen products from the dehydrogenation reaction such as the UOP Styro-Plus and Smart processes. These technologies still rely on cofeed of steam when oxygen or air are used as the oxidants for selective hydrogen combustion from dehydrogenation. Co-feeding gaseous oxygen can incur safety concerns and air separation can be expensive. Moreover, product selectivity and effectiveness of heat integration can be limited in these cases. These drawbacks also exist in the dehydrogenation of cumene to α-methylstyrene, and more generally to dehydrogenation of other alkyl aromatic compounds, due to the similarity of the two processes. Thus, the typical industrial applications have to use a second hydrogen (or other fuel) combustion reactor, high steam/feedstock ratios to offset the heat for the endothermic reaction, and complex reaction systems for heat management or products separation, leading to the loss of efficiency, high running cost, high energy consumption, as well as high carbon emissions.

We have previously demonstrated, in U.S. Pat. No. 10,946,365B2, the redox-ODH (R-ODH) using a selective hydrogen combustion catalyst for simultaneous or sequential dehydrogenation of alkylbenzenes by the catalytic dehydrogenation component (DH component) and combustion of H₂ to eliminate the aforementioned equilibrium limitations by the selective hydrogen combustion component (SHC component). As shown in the FIG. 1(a), the redox-ODH mode are operated in two steps: In the dehydrogenation step, alkylaromatics are injected to the catalyst bed, where they are dehydrogenated by the DH component, forming gaseous H₂ and unsaturated aromatics counterparts. The gaseous H₂ could be simultaneously or sequentially combusted by the lattice oxygen in the SHC component. In the regeneration step, oxidant gas (e.g., air) is injected to regenerate the selective hydrogen combustion catalyst, replenishing lattice oxygen. Although the results are highly promising, a potential challenge for these catalysts, when operated with high alkylaromatics feed pressure and steam-free conditions, is the formation of CO₂. At the initial stage of injection alkylaromatics feed, deep oxidation would likely occur. The presence of CO₂ would slowly deactivate the catalysts without optimized catalyst compositions, proper regeneration of the catalyst, and/or a tailored operating scheme to inhibit deactivation.

Preventing deep oxidation of alkylaromatics to CO₂ would optimize selectivity to desired products and preserve valuable feedstocks. The minimization of the COₓ selectivity could also minimize the formation of unwanted carbonate, which would prolong the catalyst lifetime. Herein, we disclose several specific families of SHC materials doped with rare earth metals, alkali/alkali earth metals, and/or transition metals. Doping SHC materials with other metals could have some/all of several functions: 1) Dopants could incorporate into the SHC material structures and stabilize the SHC or DH materials, reducing unselective lattice oxygen at the surface; 2) Dopants could form a uniform oxides/carbonates/chloride/nitride/boride/iodide surface layer on the SHC material core which decreases the unselective lattice oxygen concentration at the gas-solid interface; 3) Aforementioned surface layer in 2) could facilitate the formation and transport of the selective oxygen species, such as O⁻, O₂²⁻, O₂⁻ or O. As such, the resulting redox catalysts (doped SHC+DH components) could decrease the selectivity of COₓ and increase the selectivity towards the desired unsaturated aromatics. Dopants such as alkali metal oxides could also act as a sacrificing material which absorb CO₂ to prevent DH component deactivation. We also disclose several families of dopants could be applied to the DH component including rare earth metals, alkali/alkali earth metals or transition metals. Dopants could diffuse into the dehydrogenation catalyst structure to increase the resistance to CO₂. The resulting DH component could catalyze the reaction shown in Reaction (I), using dialkylbenzene as an example. The catalysts could also catalyze other aromatics, including but not limited to all alkylated benzenes, alkylated thiophenes, alkylated furans and their alkylated oligomers.

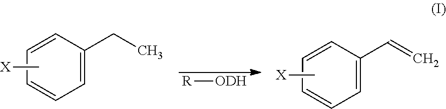

### SUMMARY

According to an exemplary embodiment of the invention, a redox catalyst comprises: a catalytic dehydration (DH) component, a COₓ management (mitigation and/or resistance) component, and an optional enhanced selective hydrogen combustion (SHC) function within the COₓ management component or an optional SHC component. The redox catalyst comprises one of the optional enhanced SHC function or the optional SHC component. The DH component comprises a first metal (iron and/or cerium)-containing oxide. The COₓ management component can be any of (i) a first dopant, wherein the DH component comprises the first dopant, and the first dopant comprises a cation of a second metal; (ii) a CO₂ sorption (COS) component, wherein the COS component comprises an oxide and/or hydroxide of a first mixed-metal, wherein the oxide and/or hydroxide of the first mixed-metal is capable of reacting with CO₂ to form the corresponding oxycarbonate and/or carbonate; and/or (iii) the optional enhanced SHC function comprising a second mixed-metal oxide comprising reactive lattice oxygen, and (a) a perovskite site component, wherein the second mixed-metal oxide comprises the perovskite site component, (b) optionally a selectivity enhancing shell on the outside of the enhanced SHC function, wherein the shell comprises oxides, carbonates, sulfates, phosphates, pyrophosphates, molybdates, tungstates, nitrates, nitride, chloride, bromide, and/or iodide of a third metal, and/or (c) a second dopant, wherein the enhanced SHC function comprises the second dopant and the second dopant comprises a carbonate and/or oxide of a fourth metal. The optional SHC component comprises the second mixed-metal oxide. The first metal, the second metal, the third metal, and/or the fourth are independently selected and can be the same or different.

According to another exemplary embodiment of the invention, a process for producing an olefinic compound (e.g., an alkenylaromatic) is provided. The process comprises the following steps: (a) providing the inventive redox catalyst in a reactor; (b) optionally an oxidized redox catalyst pre-reduction step comprising introducing hydrogen into the reactor to produce steam and a pre-reduced redox catalyst, followed by stopping introducing the hydrogen; (c) a redox catalyst dehydrogenation and selective hydrogen combustion step comprising, introducing a dehydrogenation reactant into the reactor; (d) removing the olefinic compound and the steam from the reactor, (e) stopping introducing the dehydrogenation reactant, (f) a redox catalyst regeneration step comprising, introducing an oxygen source into the reactor, wherein the reduced second mixed-metal oxide is re-oxidized to the second mixed-metal oxide, (g) stopping introducing the oxygen source, and (h) a cycle repeating step comprising repeating steps (b) through (g) at least once. During the redox catalyst dehydrogenation and selective hydrogen combustion step, (c)(i) the dehydrogenation reactant contacts the DH component to produce the olefinic compound and hydrogen, and (c)(ii) the hydrogen contacts the optional enhanced SHC function or the optional SHC component to produce steam and a reduced second mixed-metal oxide, wherein at least 10% of the hydrogen released in step c(i) is converted to steam.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 (a) is a schematic of a non-limiting embodiment of a fixed-bed reactor with redox induced oxidative dehydrogenation (R-ODH); (b) is a schematic of the fixed-bed reactor using R-ODH with a pre-reduction step using H₂ injection; (c) is a schematic of fixed-bed reactor embodiment of R-ODH with CO₂ sorption;

FIG. 2 is two schematics of non-limiting embodiments of redox catalyst configurations, (a) is an idealized representation of direct mixing of the selective hydrogenation mixed-metal oxide, the CO₂ sorption metal oxide or hydroxide and the dehydrogenation metal (iron and/or cerium)-containing oxide and (b) is an idealized representation of the dehydrogenation metal (iron and/or cerium)-containing oxide being in separate particles from the selective hydrogenation mixed-metal oxide, and the CO₂ sorption metal oxide or hydroxide, with the particles stacked in layers;

FIG. 3 is a schematic of a non-limiting embodiment of an enhanced selective hydrogen combustion function with a mixed-metal oxide core and a selectivity enhancing shell; and

FIG. 4 is a graph of the thermogravimetric analysis of Example 7.

### DETAILED DESCRIPTION

According to an exemplary embodiment of the invention, a redox catalyst comprises: a catalytic dehydration (DH) component, a COₓ management (mitigation and/or resistance) component, and an optional enhanced selective hydrogen combustion (SHC) function within the COₓ management component or an optional SHC component. The redox catalyst comprises one of the optional enhanced SHC function or the optional SHC component. The DH component comprises a first metal (iron and/or cerium)-containing oxide. The COₓ management component can be any of (i) a first dopant, wherein the DH component comprises the first dopant, and the first dopant comprises a cation of a second metal; (ii) a CO₂ sorption (COS) component, wherein the COS component comprises an oxide and/or hydroxide of a first mixed-metal, wherein the oxide and/or hydroxide of the first mixed-metal is capable of reacting with CO₂ to form the corresponding oxycarbonate and/or carbonate; and/or (iii) the optional enhanced SHC function comprising a second mixed-metal oxide comprising reactive lattice oxygen, and (a) a perovskite site component, wherein the second mixed-metal oxide comprises the perovskite site component, (b) a selectivity enhancing shell on the outside of the enhanced SHC function, wherein the shell comprises oxides, carbonates, sulfates, phosphates, pyrophosphates, molybdates, tungstates, nitrates, nitride, chloride, bromide, and/or iodide of a third metal, and/or (c) a second dopant, wherein the enhanced SHC function comprises the second dopant and the second dopant comprises a carbonate and/or oxide of a fourth metal. The optional SHC component comprises the second mixed-metal oxide. The first metal, the second metal, the third metal, and/or the fourth metal are independently selected and can be the same or different.

The present invention may be understood more readily by reference to the following detailed description of the invention taken in connection with the accompanying drawing figures, which form a part of this disclosure. It is to be understood that this invention is not limited to the specific devices, methods, conditions, or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

It is to be understood that the mention of one or more method steps does not preclude the presence of additional method steps before or after the combined recited steps or intervening method steps between those steps expressly identified. Moreover, the lettering of method steps or ingredients is a conventional means for identifying discrete activities or ingredients and the recited lettering can be arranged in any sequence, unless otherwise indicated.

As used herein, the term “and/or”, when used in a list of two or more items, means that any one of the listed items can be employed by itself, or any combination of two or more of the listed items can be employed. For example, if a composition is described as containing compounds A, B, “and/or” C, the composition may contain A alone; B alone; C alone; A and B in combination; A and C in combination; B and C in combination; or A, B, and C in combination.

As used herein, the term “dehydrogenation component” or “DH component” refers to a mixed-metal oxide with a first metal and iron and/or cerium. The mixed (iron and/or cerium)-containing oxide promotes the oxidative dehydration reaction of alkylaromatics. The “dehydrogenation component” may also comprise a dopant, which is a cation of a metal, typically included in an ionic compound added to the mixed metal oxide.

As used herein, the term “COₓ management component” refers to any of the catalyst features that are designed to either mitigate (i.e., reduce the formation of CO or CO₂) or resist (i.e., protect the “dehydration component” mixed (iron and/or cerium)-containing oxide from CO or CO₂ poisoning and/or provide for reactive capture of the CO₂ in a “CO₂ sorption component”).

As used herein, the term “CO₂ sorption component” or “COS component” refers to an oxide and/or hydroxide of two metals capable of reacting with CO₂ to form the corresponding carbonate or hydrate. “CO₂ sorption component” is typically physically separate (even if part of the same particle) from the “dehydration component”. When the catalyst is regenerated, an oxygen source reacts with the carbonate and/or hydrate to reform CO₂ which is removed from the catalyst. The “CO₂ sorption component” may be separate or may be mixed (at atomistic scales) with the mixed-metal oxide of the “selective hydrogen combustion component” or “enhanced selective hydrogen combustion function”.

As used herein, the term “selective hydrogen combustion component” or “SHC component” refers to a mixed-metal oxide capable of selectively reacting with the hydrogen released in the oxidative dehydrogenation step to form steam. As used herein, the term, “enhanced selective hydrogen combustion function” or “enhanced SHC function” refers to the mixed-metal oxide capable of selectively reacting with the hydrogen released in the oxidative dehydrogenation step to form steam, wherein the mixed-metal oxide has been enhanced to improve the selectivity of the hydrogen combustion. Example enhancements include, but are not limited to, partial substitution of the mixed metal site in a perovskite oxide, (e.g., LaₓCa₁₋ₓMnO₃), a selectivity enhancing shell on the outside of the mixed-metal oxide, or a dopant mixed with the mixed-metal oxide. The redox catalyst comprises one of the “selective hydrogen combustion component” or the “enhanced selective hydrogen combustion function”. The “enhanced selective hydrogen combustion function within the ‘COₓ management component’” refers to the classification of the “enhanced selective hydrogen combustion function” as a one of the options for the “COₓ management component”.

As used herein, the term “perovskite site component” refers to the metal that partially substitutes the A or the B site of a perovskite oxide. For example, (LaₓCa₁₋ₓ)_{y} MnO₃ represents perovskite oxide of the formula ABO₃, wherein the A site is partially substituted. Ca is the “perovskite site component” if x is greater than 0.5, and La is the “perovskite site component if x is less than 0.5. Y is an optional notation to represent modifications to the active lattice oxygen amount.

As used herein, the term “K—Fe—O”, refers to any oxide consisting of potassium, iron, and oxygen. Examples include, but are not limited to, mixtures of K₂O/Fe₂O₃, and compounds such as KFeO₂, K₂FeO₄, and KFe₅O₈. The analogy applies also to Li—Fe—O, Na—Fe—O, and Zn—Fe—O. Throughout the description and claims when a catalyst is described as a set of oxides, e.g., La₂O₃/CaO/MnO₂, the catalyst includes simple mixtures of the oxides and/or any reaction products formed during the catalyst production process and can simply be described as an oxide consisting of La, Ca, and Mn.

The configuration of redox catalyst comprising the catalytic dehydration (DH) component, the COₓ management component and the optional SHC function or the optional SHC component can vary. In some aspects, the configuration of the redox catalyst is a single particle comprising the DH component, the optional enhanced SHC function, the optional SHC component and/or the COS component. In some aspects, the configuration of the redox catalyst is multiple particles wherein the DH component is in a distinct particle from the optional enhanced SHC function, the optional SHC component, and/or COS component. In some aspects, the configuration of the redox catalyst is multiple particles wherein each particle independently comprises the DH component, the optional enhanced SHC function, the optional the SHC component, and/or the COS component. In some aspects, the configuration of the redox catalyst is the single particle, wherein the DH component, optional enhanced SHC function, the optional SHC component, and/or COS component form two or more distinct phases, and the phases are arranged in a core-shell, wherein the outermost shell comprises the DH component. In some aspects, the redox catalyst further comprises structured material comprising graphite and/or boron nitride

The redox comprises a first metal (iron and/or cerium)-containing oxide in the dehydration (DH) component. In some aspects, the first metal is selected from the group consisting of alkali metals. In some aspects, the first metal (iron and/or cerium)-containing oxide is selected from the group consisting or (i) a potassium iron-containing oxide selected from the group consisting of K—Fe—O, K₂O/MeFe₂O₄, and/or K₂O/Ca₂Fe₂O₅, or a potassium iron-containing-oxide comprising K—Fe—O, mixed K-Mₓ oxides, mixed Fe-Mₓ oxides, and/or mixed K—Fe-Mₓ oxides, wherein Mₓ is selected from the group consisting of Ca, Mo, Mn, and/or Cr; (ii) a lithium iron-containing oxide selected from the group consisting of Li—Fe—O, Li₂O/MeFe₂O₄, and/or Li₂O/Ca₂Fe₂O₅; (iii) a sodium iron-containing oxide selected from the group consisting of Na—Fe—O, Na₂O/MeFe₂O₄, and/or Na₂O/Ca₂Fe₂O₅; (iv) a zinc iron-containing oxide selected from the group consisting of Zn—Fe—O, Zn₂O/MeFe₂O₄, and/or Zn₂O/Ca₂Fe₂O₅; and/or a (v) cerium-containing-oxide selected from the group consisting of K—Ce—O, Li₂O/CeO₂, Na₂O/CeO₂, and/or Zn₂O/CeO₂, wherein Me is independently selected from the group consisting of Mn, Cu, Co, Zn, and/or Ni.

In some aspects, the first metal (iron and/or cerium)-containing oxide comprises a potassium iron-containing oxide. In some aspects a weight ratio of K₂O to Fe₂O₃ ranges from 1:10 to 1:1, or from 1:10 to 1:2, or from 1:10 to 1:4, or from 1:10 to 1:6, or from 1:10 to 1:8, or from 1:8 to 1:1, or from 1:8 to 1:2, or from 1:8 to 1:4, or from 1:8 to 1:6, or from 1:6 to 1:1, or from 1:6 to 1:2, or from 1:6 to 1:4, or from 1:4 to 1:1 or from 1:4 to 1:2, or from 1:2 to 1:1. In some aspects, a mole ratio of K to Fe ranges from 0.5:1 to 2:1, or from 0.5:1 to 1:1.5, or from 0.5:1 to 1:1, or from 1:1 to 2:1, or from 1:1 to 1.5. In some aspects, the first metal (iron and/or cerium)-containing oxide comprises a potassium cerium-containing oxide. In some aspects a weight ratio of K₂O to CeO₂ ranges from 1:10 to 1:1, or from 1:10 to 1:2, or from 1:10 to 1:4, or from 1:10 to 1:6, or from 1:10 to 1:8, or from 1:8 to 1:1, or from 1:8 to 1:2, or from 1:8 to 1:4, or from 1:8 to 1:6, or from 1:6 to 1:1, or from 1:6 to 1:2, or from 1:6 to 1:4, or from 1:4 to 1:1 or from 1:4 to 1:2, or from 1:2 to 1:1, or from 1:6 to 2:1, or from 1:4 to 2:1, or from 1:2 to 2:1 or from 1:1 to 2:1. In some aspects a mole ratio of K to Ce ranges from 0.5:1 to 2:1, or from 0.5:1 to 1:1.5, or from 0.5:1 to 1:1, or from 1:1 to 2:1, or from 1:1 to 1.5.

The redox catalyst comprises one of the optional enhanced selective hydrogen combustion (SHC) function or the SHC component. Either the optional SHC function or the optional SHC component comprises a second mixed-metal oxide. In some aspects, the second mixed-metal is selected from the group consisting of alkaline earth metals, transition metals, and/or lanthanide metals. In some aspects, the second mixed-metal oxide is selected from the group consisting of (i) an iron-containing oxide, wherein the iron-containing oxide optionally comprises an alkaline earth metal and/or a transition metal other than iron; (ii) a vanadium oxide; (iii) bismuth molybdate or molybdenum oxide; and/or (iv) perovskite oxides or oxides selected from the group consisting of ABO₃, A₂B₂O₅, and/or Aₙ₊₁BₙO₃ₙ₊₁, wherein A is selected from the group consisting of alkaline earth metals and/or lanthanide metals, and B is selected from the group consisting of transition metals and/or lanthanide metals, and wherein n is a positive integer or wherein n is 1, 2, 3, or 4. In some aspects, the second mixed-metal oxide is selected from the group consisting of (i) the iron-containing oxide, wherein the iron-containing oxide is selected from the group consisting of Fe₂O₃, Fe₃O₄, MnFe₂O₄, CuFe₂O₄, ZnFe₂O₄, I₂O₄, and/or MgFe₂O₄; (ii) a vanadium oxide; (iii) bismuth molybdate or molybdenum oxide; and/or (iv) an oxide selected from the group consisting of ABO₃, A₂B₂O₅, and/or Aₙ₊₁BₙO₃ₙ₊₁, wherein A is selected from the group consisting of Ca, Sr, Ba, and/or LA and B is selected from the group consisting of Zn, Mn, Fe, Co, and/or Pr. In some aspects, A is selected from the group consisting or Ca, Zn, La, Y, Mg, Na, K and B is selected from the group consisting of transition metals.

In some aspects, the second mixed oxide comprises a perovskite site component. In some aspects, the perovskite site component is selected from the group consisting of alkaline earth metal and/or lanthanide metals. In some aspects, the second mixed-metal oxide comprises ABO₃, A₂B₂O₅, and/or Aₙ₊₁BₙO₃ₙ₊₁, and at least one of the A or the B site metals is partially substituted with the perovskite site component. In some aspects, (i) the A site metal is selected from the group consisting of alkaline earth metals and/or lanthanide metals; or wherein the A site metal is selected from the group consisting of Ca, Zn, La, Y, Mg, Na, and/or K; (ii) the B site metal is selected from the group consisting of transition metals; or wherein the B site metal is selected from the group consisting of Mn, Fe, and/or Co; and (iii) the perovskite site component is selected from the group consisting of alkaline earth metals, transition metals, and/or lanthanide metals; or wherein the perovskite site component is selected from the group consisting of Ca, Zn, La, Y, Mg, Na, K, Mn, Fe, and/or Co. In some aspects, the partially substituted A site is represented by (A¹ₓA²₁₋ₓ), wherein 0.01<x<0.99 or 0.05<x<0.95 or 0.1<x<0.9 or 0.15<x<0.85, wherein if x>0.5, the perovskite site component is A¹ and if x<0.5, the perovskite site component is A². In some aspects, the partially substituted B site is represented by (B¹ₓB²₁₋ₓ), wherein 0.01<x<0.99 or 0.05<x<0.95 or 0.1<x<0.9 or 0.15<x<0.85, wherein if x>0.5, the perovskite site component is B¹ and if x<0.5, the perovskite site component is B².

The redox catalyst comprises a catalytic dehydration (DH) component, a COₓ management (mitigation and resistance) component, and an optional enhanced SHC function within the COₓ management component or an optional SHC component. In some aspects, the COₓ management component comprises a first dopant, wherein the DH component comprises the first dopant, and the first dopant comprises a cation of a second metal. In some aspects, the second metal is selected from the group consisting of alkali metals and/or alkaline earth metals, transition metals, post-transition metals, and/or lanthanide metals. In some aspects, the second metal is selected from the group consisting of Ag, Bi, Ca, Ce, Cr, Li, Ga, Mg, Mo, Mn, Ti, V and/or Zr, and an ionic compound comprising the cation of the second metal is selected from the group consisting of oxides, carbonates, sulfates, phosphates, pyrophosphates, molybdates, tungstates, nitrates. In some aspects, second metal is selected from the group consisting of Ag, Bi, Ce, Cr, Ga, and/or Mo, and wherein an ionic compound comprising the cation of the second metal is selected from the group consisting of oxides, carbonates, sulfates, phosphates, pyrophosphates, molybdates, tungstates, and/or nitrates. In some aspects, the second metal is selected from the group consisting of Mg, Bi, Ce, Cr, Ti, Mo, and/or Zr. In some aspects, the second metal is selected from the group consisting of Mg, Bi, Ce, Cr, Ti, Mo, and/or Zr and the ionic compound comprises MgO/MoO₃/AgO; MgO/Cr₂O₃/AgO; MgO/Cr₂O₃/CeO₂; MgO/MoO₃/ZrO₂; MgO/MoO₃/Bi₂O₃; MgO/ZrO₂/Bi₂O₃; and/or MgO/CeO₂/Bi₂O₃. In some aspects, the ionic compound is represented as A/B/C and has a weight ratio A:B ranging from 1:0.01 to 0.01:1 and a weight ratio of B:C ranging from 1:0.01 to 0.01:1. In some aspects, the DH component comprises the ionic compound in an amount ranging from 0.1 wt. % to 40 wt. %, or from 0.1 wt. % to 30 wt. %, or from 0.1 wt. % to 20 wt. %, or from 0.1 wt. % to 15 wt. %, or from 0.1 wt. % to 10 wt. %, or from 0.1 wt. % to 7 wt. %, or from 0.1 wt. % to 5 wt. %, or from 0.1 wt. % to 3 wt. %, or from 0.1 wt. % to 1 wt. %, or from 1 wt. % to 40 wt. %, or from 1 wt. % to 30 wt. %, or from 1 wt. % to 20 wt. %, or from 1 wt. % to 15 wt. %, or from 1 wt. % to 10 wt. %, or from 1 wt. % to 7 wt. %, or from 1 wt. % to 5 wt. %, or from 1 wt. % to 3 wt. %, or from 3 wt. % to 40 wt. %, or from 3 wt. % to 30 wt. %, or from 3 wt. % to 20 wt. %, or from 3 wt. % to 15 wt. %, or from 3 wt. % to 10 wt. %, or from 3 wt. % to 7 wt. %, or from 3 wt. % to 5 wt. %, or from 5 wt. % to 40 wt. %, or from 5 wt. % to 30 wt. %, or from 5 wt. % to 20 wt. %, or from 5 wt. % to 15 wt. %, or from 5 wt. % to 10 wt. %, or from 10 wt. % to 40 wt. %, or from 10 wt. % to 30 wt. %, or from 10 wt. % to 20 wt. %, or from 10 wt. % to 15 wt. %, based upon a total weight of the DH component.

In some aspects, the COₓ management component comprises a CO₂ sorption (COS) component, wherein the COS component comprises an oxide and/or hydroxide of a first mixed-metal. In some aspects the first mixed-metal is selected from the group consisting of alkali metals, alkaline earth metals, and/or transition metals. In some aspects, the first mixed-metal is selected from the group consisting of Li, Na, K, V, Mn, Fe, Co, and/or Cu. In some aspects, the first mixed-metal oxide or hydroxide comprises K and a metal selected from the group consisting of Li, Na, V, Mn, Fe, Co, and/or Cu. In some aspects, the first mixed-metal oxide and/or hydroxide comprises K₂O in a range from 5 wt. % to 75 wt. %, or from 10 wt. % to 60 wt. %, or from 25 wt. % to 50 wt. % on a total first mixed-metal oxide weight basis. In some aspects, the molar ratio of the DH component to the COS component, the ratio of the moles of the first metal (iron and/or cerium)-containing oxide to the first mixed-metal oxide, ranges from 50:1 to 1:1 or from 40:1 to 1:1 or from 30:1 to 1:1 or from 20:1 to 1:1 or from 10:1 to 1:1 or from 5:1 to 1:1.

In some aspects, the COₓ management component comprises the optional enhanced SHC function comprising a second mixed-metal oxide comprising reactive lattice oxygen, and a selectivity enhancing shell on the outside of the enhanced SHC function, wherein the shell comprises oxides, carbonates, sulfates, phosphates, pyrophosphates, molybdates, tungstates, nitrates, nitride, chloride, bromide, and/or iodide of a third metal. In some aspects, third metal is selected from the group consisting of alkali metals, alkaline earth metals, and/or rare earth metals. In some aspects, the third metal is selected from the group consisting of Li, Na, K, Rb, Cs, Be, Mg, Ca, Sr, and/or Ba. In some aspects, the third metal is selected from the group consisting of Li, Na, and/or K. In some aspects, the third metal is selected from the group consisting of Li, Na, and/or, K, and the selectivity enhancing shell comprises the oxide K₂CO₃/Li₂CO₃/Na₂CO₃. In some aspects, a weight percent of the selectivity enhancing shell the based on the total weight of the enhanced SHC function ranges from 1 wt. % to 20 wt. %, or from 1 wt. % to 15 wt. %, or from 1 wt. % to 10 wt. %, or from 1 wt. % to 7.5 wt. %, or from 1 wt. % to 5 wt. %, or from 2.5 wt. % to 20 wt. %, or from 2.5 wt. % to 15 wt. %, or from 2.5 wt. % to 10 wt. %, or from 2.5 wt. % to 7.5 wt. %, or from 2.5 wt. % to 5 wt. %, or from 5 wt. % to 20 wt. %, or from 5 wt. % to 15 wt. %, or from 5 wt. % to 10 wt. %, or from 5 wt. % to 7.5 wt. %, or from 7.5 wt. % to 20 wt. %, or from 7.5 wt. % to 15 wt. %, or from 7.5 wt. % to 10 wt. %, or from 10 Wt. % to 20 wt. %, or from 10 wt. % to 15 wt. %. In some aspects, the molar ratio of K:(Li+Na) ranges from 0.01:1 to 1:0.01.

In some aspects, the COₓ management component comprises the optional enhanced SHC function comprising a second mixed-metal oxide comprising reactive lattice oxygen and a second dopant. In some aspects, the enhanced SHC function comprises the second dopant. In some aspects, the second dopant comprises a carbonate and/or oxide of a fourth metal. In some aspects, the fourth metal is selected from the group consisting of alkali metals and/or alkali earth metals.

In some aspects, the first metal (iron and/or cerium)-containing oxide comprises K₂O/Fe₂O₃, the redox catalyst comprises the optional enhanced SHC function and the second mixed-metal oxide comprises La₂O₃/CaO/MnO₂, the perovskite site component is selected from the group consisting of La or Ca, the enhanced SHC function optionally comprises the second dopant comprising K₂CO₃ in an amount ranging from 0 wt. % to 10 wt. % based on the total weight of the enhanced SHC function, and the DH component optionally comprises the first dopant comprising MgO/MoO₃/ZrO₂ in an amount ranging from 0 to 5 wt. % based on the total weight of the DH component.

In some aspects, the first metal (iron and/or cerium)-containing oxide comprises K₂O/CeO₂, the redox catalyst comprises the optional enhanced SHC function and the second mixed-metal oxide comprises La₂O₃/CaO/Fe₂O₃, the perovskite site component is selected from the group consisting of La or Ca, the enhanced SHC function optionally comprises the second dopant comprising Cs₂CO₃/K₂CO₃ in an amount ranging from 0 wt. % to 10 wt. % based on the total weight of the enhanced SHC function, and the DH component optionally comprises the first dopant comprising MgO/Cr₂O₃/AgO in an amount ranging from 0 to 7 wt. % based on the total weight of the DH component.

In some aspects, the first metal (iron and/or cerium)-containing oxide comprises K₂O/Fe₂O₃, the redox catalyst comprises the optional enhanced SHC function and the second mixed-metal oxide comprises La₂O₃/ZnO/Fe₂O₃, the perovskite site component is selected from the group consisting of La or Zn, the enhanced SHC function optionally comprises the second dopant comprising MgO/Na₂CO₃ in an amount ranging from 0 wt. % to 10 wt. % based on the total weight of the enhanced SHC function, the DH component optionally comprises the first dopant comprising MgO/Cr₂O₃/CeO₂ in an amount ranging from 0 to 7 wt. % based on the total weight of the DH component.

In some aspects, the first metal (iron and/or cerium)-containing oxide comprises K₂O/Fe₂O₃, the redox catalyst comprises the optional enhanced SHC function and the second mixed-metal oxide comprises La₂O₃/CaO/MnO₂, the perovskite site component is selected from the group consisting of La or Ca, the selectivity enhancing shell comprises K₂CO₃/Li₂CO₃/Na₂CO₃ and a weight percent of the selectivity enhancing shell based on the total weight of the enhanced SHC function ranges from 1 wt. % to 20 wt. %, and the DH component optionally comprises the first dopant comprising MgO/MoO₃/ZrO₂ in an amount ranging from 0 to 5 wt. % based on the total weight of the DH component.

In some aspects, the first metal (iron and/or cerium)-containing oxide comprises K₂O/CeO₂, the redox catalyst comprises the optional enhanced SHC function and the second mixed-metal oxide comprises ZnO/Fe₂O₃, the selectivity enhancing shell comprises K₂CO₃/Li₂CO₃/Na₂CO₃ and a weight percent of the selectivity enhancing shell based on the total weight of the enhanced SHC function ranges from 1 wt. % to 20 wt. %, and the DH component optionally comprises the first dopant comprising MgO/MoO₃/Bi₂O₃ in an amount ranging from 0 to 5 wt. % based on the total weight of the DH component.

In some aspects, the first metal (iron and/or cerium)-containing oxide comprises K₂O/CeO₂, the redox catalyst comprises the optional enhanced SHC function and the second mixed-metal oxide comprises a Ruddlesden-Popper structure, (CaₓA₁₋ₓ)ₙ₊₁((Fe/Mn)_{y}B_{1-y})ₙO₃ₙ₊₁, wherein A is selected from the group of metals consisting of La, Sr, Ce, Ba, Pr, Sm, or Y, B is selected from the group of metals consisting of Co, Ti, Mg, Zr, Ce, Cr, or Mo, x ranges from 0 to 1, y ranges from 0 to 1, and n can be 1, 2, 3 or 4, the selectivity enhancing shell comprises K₂CO₃/Li₂CO₃/Na₂CO₃ and a weight percent of the selectivity enhancing shell based on the total weight of the enhanced SHC function ranges from 1 wt. % to 20 wt. %, and the DH component optionally comprises the first dopant comprising MgO/MoO₃/ZrO₂ in an amount ranging from 0 to 5 wt. % based on the total weight of the DH component.

In some aspects the first metal (iron and/or cerium)-containing oxide comprises K₂O/Fe₂O₃, the redox catalyst comprises the optional enhanced SHC function and the second mixed-metal oxide comprises CaO/La₂O₃/MnO₂/CeO₂, wherein the perovskite site component is selected from the group consisting of La and Ca, the DH component comprises the first dopant comprising MgO/ZrO₂/Bi₂O₃ in an amount ranging from 1 to 5 wt. % based on the total weight of the DH component, and the redox catalyst comprises the COS component, wherein the first mixed-metal oxide comprises K₂O/MnO₂.

In some aspects, the first metal (iron and/or cerium)-containing oxide comprises K₂O/Fe₂O₃, the redox catalyst comprises the optional enhanced SHC function and the second mixed-metal oxide comprises MgO/MnO₂/Fe₂O₃, the DH component comprises the first dopant comprising MgO/CeO₂/Bi₂O₃ in an amount ranging from 1 to 5 wt. % based on the total weight of the DH component, and the redox catalyst comprises the COS component, wherein the first mixed-metal oxide comprises Li₂O/MnO₂. In some aspects, the redox catalyst further comprises structured material comprising graphite and/or boron nitride.

In some aspects, the redox catalyst comprises the optional enhanced SHC function and the enhanced SHC function comprises CaO, Fe₂O₃, MoO₃, AgO, Cr₂O₃, MgO and/or CeO₂ containing oxides, and an amount of CaO range from 2 wt. % to 15 wt. %, an amount of Fe₂O₃ ranges from 50 wt. % to 85 wt. %, an amount of MoO₃ ranges from 0 wt. % to 2 wt. %, an amount of AgO ranges from 0 wt. % to 2 wt. %, an amount of Cr₂O₃ ranges from 0 wt. % to 5 wt. %, an amount of MgO ranges from 0 wt. % to 10 wt. % and an amount of CeO₂ ranges from 1 wt. % to 10 wt. %.

In some aspects, a molar ratio of the first metal (iron and/or cerium)-containing oxide to the second mixed-metal oxide ranges from 1:10 to 10:1, or from 1:5 to 5:1.

In some aspects, the redox catalyst further comprises a heat transfer agent, wherein the heat transfer agent comprises an inert compound. In some aspects, the inert compound comprises Al₂O₃, ZrO₂, SiC and/or TiO₂. In some aspects, the redox catalyst and the heat transfer agent are separate particles. In some aspects, the redox catalyst and the heat transfer agent are configured in a core-shell with the heat transfer agent as the core.

According to another exemplary embodiment of the invention, a process for producing an olefinic compound (e.g., an alkenylaromatic) is provided. The process comprises the following steps: (a) providing the inventive redox catalyst in a reactor; (b) optionally an oxidized redox catalyst pre-reduction step comprising introducing hydrogen into the reactor to produce steam and a pre-reduced redox catalyst, followed by stopping introducing the hydrogen; (c) a redox catalyst dehydrogenation and selective hydrogen combustion step comprising, introducing a dehydrogenation reactant into the reactor; (d) removing the olefinic compound and the steam from the reactor, (e) stopping introducing the dehydrogenation reactant, (f) a redox catalyst regeneration step comprising, introducing an oxygen source into the reactor, wherein the reduced second mixed-metal oxide is re-oxidized to the second mixed-metal oxide, (g) stopping introducing the oxygen source, and (h) a cycle repeating step comprising repeating steps (b) through (g) at least once. During the redox catalyst dehydrogenation and selective hydrogen combustion step, (f) (i) the dehydrogenation reactant contacts the DH component to produce the olefinic compound and hydrogen, and (f) (ii) the hydrogen contacts the optional enhanced SHC function or the optional SHC component to produce steam and a reduced second mixed-metal oxide, wherein at least 10% of the hydrogen released in step c(i) is converted to steam.

In some aspects, the dehydrogenation reactant comprises an alkyl aromatic hydrocarbon or a substituted alkyl aromatic hydrocarbon and the olefinic compound comprises an alkene aromatic hydrocarbon or substituted alkene aromatic hydrocarbon, respectively. In some aspects, the dehydrogenation reactant comprises alkylated benzenes, alkylated thiophenes, alkylated furans, oligomers of alkylated benzenes, oligomers of alkylated thiophenes, and/or oligomers of alkylated furans. In some aspects, the dehydrogenation reactant comprises ethylbenzene, di-ethylbenzene, diisopropylbenzene, and/or cumene. In some aspects, the dehydrogenation reactant comprises 1,2-diisopropylbenzene, 1,3-diisopropylbenzene and/or 1,4-diisopropeylbenzene, and the olefinic compound comprises 1,2-diisopropenylbenzene, 1,3-diisopropenylbenzene and/or 1,4-diisopropenylbenzene.

In some aspects, the reactor temperature during the redox catalyst dehydrogenation and selective hydrogen combustion step (c) ranges from 350° C. to 900° C., or from 400° C. to 700° C., or from 450° C. to 650° C. In some aspects, the redox catalyst dehydrogenation and selective hydrogen combustion step (c) occurs for a time range of 5 minutes to 30 minutes, or at a time range of 5 minutes to 60 minutes, or at a time range of 5 minutes to 120 minutes. In some aspects at least 15% of the hydrogen released in step c(i) is converted to steam. Other non-limiting examples of the amount of the hydrogen released in step c(i) which is converted to steam is at least 20%, or at least 25%, or at least 30%, or at least 40%, or at least 50%.

In some aspects, the dehydrogenation reactant contacts the redox catalyst for a contact time ranging from 0.1 s to 2,000 s, or from 0.1 s to 1,000 s, or from 0.1 s to 500 s, or from 0.1 sec to 250 s, or from 0.1 s to 120 s, or from 0.1 s to 60 s, or from 10 s to 2,000 s, or from 10 s to 1,000 s, or from 10 s to 500 s, or from 10 sec to 250 s, or from 10 s to 120 s, or from 10 s to 60 s, or from 30 s to 2,000 s, or from 30 s to 1,000 s, or from 30 s to 500 s, or from 30 s to 250 s, or from 30 s to 120 s, or from 30 s to 60 s.

In some aspects, wherein the oxygen source of the redox catalyst regeneration step (f) comprises air, steam, CO₂, and/or O₂ rich gas. In some aspects, the redox catalyst regeneration step (f) occurs for a time range of 5 minutes to 30 minutes, or at a time range of 5 minutes to 60 minutes, or at a time range of 5 minutes to 120 minutes.

In some aspects, the oxidized redox catalyst pre-reduction step (b) occurs, and a gas stream is introduced to the reactor. In some aspects, the gas stream comprises between 1 vol. % to 10 vol. % hydrogen and 99 vol. % to 90 vol. % inerts. In some aspects, the gas stream contacts the redox catalyst for a contact time ranging from 0.1 s to 2,000 s, or from 0.1 s to 1,000 s, or from 0.1 s to 500 s, or from 0.1 sec to 250 s, or from 0.1 s to 120 s, 0.1 s to 60 s, or from 0.1 sec to 45 s, or from 0.1 sec to 30 sec, or from 10 s to 2,000 s, or from 10 s to 1,000 s, or from 10 s to 500 s, or from 10 sec to 250 s, or from 10 s to 120 s, or from 10 s to 60 s, or from 10 s to 45 s, or from 10 sec to 30 sec. In some aspects the oxidized redox catalyst prereduction step (b) occurs for a time range of 5 minutes to 30 minutes, or at a time range of 5 minutes to 60 minutes, or at a time range of 5 minutes to 120 minutes.

In some aspects, when the cycle repeating step (h) has occurred between 10 and 1,000 time, or between 10 and 500 times, or between 10 and 250 times, or between 10 and 100 times, or between 50 and 1,000 times, or between 50 and 500 times, or between 50 and 250 times, or between 50 and 100 times: then the oxygen source introduced in the next redox catalyst regeneration step (f) comprises greater than 50 vol. % O₂, or greater than 60 vol. % O₂, or greater than 70 vol. % O₂, or greater than 80 vol. % 02.

In some aspects, the reactor is operated in a configuration during steps (b) through (g) selected from the group consisting of fluid bed, fixed bed, moving bed, simulated moving bed, or rotating bed. In some aspects an operating pressure ranges from 0.1 bar to 5 bar.

In some aspects, steam is co-fed with the dehydrogenation reactant comprises an alkyl aromatic hydrocarbon or a substituted alkyl aromatic hydrocarbon and a mole ratio of the steam to the dehydrogenation reactant ranges from 100:1 to 1:1, or from 75:1 to 1:1, or from 50:1 to 1:1, or from 25:1 to 1:1, or from 10:1 to 1:1, or from 5:1 to 1:1. In some aspects, steam is co-fed with the dehydrogenation reactant comprises an alkyl aromatic hydrocarbon or a substituted alkyl aromatic hydrocarbon and a weight ratio of the steam to the dehydrogenation reactant ranges from 100:1 to 1:1, or from 75:1 to 1:1, or from 50:1 to 1:1, or from 25:1 to 1:1, or from 10:1 to 1:1, or from 5:1 to 1:1.

In some aspects, the introducing of the dehydrogenation reactant into the reactor in step (c) occurs in a feed stream comprising the dehydrogenation reactant, steam, and inert. In some aspects, the mole percent of dehydrogenation reactant in the feed ranges from 1 mole % to 40 mole %, or from 1 mole % to 20 mole %, or 1 mole % to 10 mole %; or from 3 mole % to 40 mole %, or from 3 mole % to 20 mole %, or 3 mole % to 10 mole %. In some aspects, the mole percent of steam in the feed stream ranges from 1 mole % to 60 mole %, or from 1 mole % to 40 mole % or from 1 mole % to 20 mole %, or 5 mole % to 60 mole %, or from 5 mole % to 40 mole % or from 5 mole % to 20 mole %, or 10 mole % to 60 mole %, or from 10 mole % to 40 mole % or from 10 mole % to 20 mole %. In some aspects, the mole percent of inert in the feed stream ranges from 20 mole % to 90 mole %, or from 20 mole % to 80 mole %, or 20 mole % to 60 mole %. In some aspects, the inert comprises nitrogen and/or argon.

In some aspects, COₓ mitigation component comprises a CO₂ sorption (COS) component, wherein the COS component comprises an oxide and/or hydroxide of a first mixed-metal. The COS component captures CO₂ co-generated by the catalyst via chemical reaction, forming the corresponding oxycarbonate or carbonate. During the catalyst regeneration step, the oxycarbonate or carbonate is converted back to CO₂. An example set of chemical reaction with the COS component comprising K₂MnO₃ is given below:

H₂+2K₂MnO₃+2CO₂→2K₂CO₃+Mn₂O₃+H₂O Reaction 1

O₂+4K₂CO₃+2Mn₂O₃→4CO₂+4K₂MnO₃ Reaction 2

In some aspects, the COS component is physically distinct from the DH component, thus keeping CO₂ and/or coke from forming on the DH component.

In some aspects, the COₓ management component comprises the optional enhanced SHC function wherein the enhanced SHC function comprises a selectivity enhancing shell around the second mixed-metal oxide. Non-limiting examples of potential selectivity enhancing shell compounds and amounts are giving in Table 1.

TABLE 1
SHC component dopants/shell composition

| Dopants | |
|---|---|
| (oxides/carbonates/ | Range |
| sulfates/phosphates/ | (mol. |
| pyrophosphates/molybdates/ | %, |
| tungstates/nitrates/nitride/chloride/ | metal |
| boride/iodide of the specified metal) | basis) |
| Li | 0~30% |
| Na | |
| K | |
| Rb | |
| Cs | |
| Be | |
| Mg | |
| Ca | |
| Sr | |
| Ba | |

In some aspects, the redox catalyst comprises a COₓ management component, wherein the DH component comprises a dopant, and the dopant comprises a cation. Non-limiting examples of ionic compounds that can supply the metal cation and amounts are given in Table 2.

TABLE 2
DH component dopants composition
K—Fe—O mixture as a DH catalyst

| Dopants | Range (wt. %) |
|---|---|
| AgO | 0.1~10% |
| Bi₂O₃ | 0.1~10% |
| CaO | 1~30% |
| CeO₂ | 0~15% |
| Cr₂O₃/CrO₃ | <1% |
| Li₂O | 1~5% |
| MgO | 0~20% |
| MoO₃ | 0~5% |
| MnO₂ | 0~20% |
| V₂O₅ | 0~20% |
| ZrO₂ | 0.1~40% |
| Ga₂O₃ | 0~5% |

Several combinations of redox catalyst comprising DH component, enhanced SHC function, and/or additional COₓ management components fall within the scope of the present invention. Given below are several non-limiting embodiments of materials and relative amounts of materials that can be used to make redox catalysts of the present invention in several different configurations.

SHC/DH composite or core-shell with SHC and SHC dopant as core and DH and DH
dopant as shell

| SHC function | SHC | DH catalyst component | |
|---|---|---|---|
| (ratio by weight) | function dopant | (ratio by weight) | DH dopant |
| La₂O₃/CaO/MnO₂ | 0~10 wt. % | K₂O/Fe₂O₃ | 0-5 wt. % |
| (La₂O₃:CaO = | K₂CO₃— | (K₂O/Fe₂O₃ = | MgO/MoO₃/AgO |
| 0.01:1~1:0.01; | | 1:10~1:1) | (MgO:MoO₃ = 1:0~0:1; |
| CaO:MnO₂ = 1:5~5:1) | | | MoO₃/AgO = 1:0~0:1) |
| La₂O₃/CaO/Fe₂O₃ | 0~10 wt. % | K₂O/CeO₂ | 0-7 wt. % |
| (La₂O₃:CaO = | Cs₂CO₃/K₂CO₃ | (K₂O/CeO₂ = | MgO/Cr₂O₃/AgO |
| 0.01:1~1:0.01; | (Cs₂CO₃:K₂CO₃ = | 1:6~2:1) | (MgO: |
| CaO:Fe₂O₃ = | 0.01:1~1:0.01) | | Cr₂O₃ = 1:0~0:1; |
| 1:7~7:1) | | | Cr₂O₃/AgO = 1:0~0:1) |
| La₂O₃/ZnO/Fe₂O₃ | 0~10 wt. % | K₂O/ Fe₂O₃ | 0-7 wt. % |
| (La₂O₃:ZnO = | MgO/Na₂CO₃ | (K₂O/Fe₂O₃ = | MgO/Cr₂O₃/CeO |
| 0.01:1~1:0.01; | (MgO:Na₂CO₃ = | 1:10~1:1) | (MgO: |
| ZnO: Fe₂O₃ = | 0.01:1~1:0.01) | | Cr₂O₃ = 1:0~0:1; |
| 1:7~7:1) | | | Cr₂O₃/CeO₂ = 1:0~0:1) |

Core-Shell SHC + DH in composite or segregated forms

| | | DH catalyst | |
|---|---|---|---|
| SHC function Core | SHC function Shell | component | DH dopant |
| La₂O₃/CaO/MnO2 | 2~20 wt. % | K₂O/Fe₂O₃ | 0-5 wt. % |
| (La₂O₃:CaO = | K₂CO₃/Li₂CO₃/Na₂CO₃ | (K₂O/Fe₂O₃ = | MgO/MoO₃/ZrO₂ |
| 0.01:1~1:0.01; | (K : (Li + Na) = | 1:10~1:1) | (MgO:MoO₃ = 1:0~0:1; |
| CaO:MnOx = 1:5~5:1) | 0.01:1~1:0.01) | | MoO₃/ZrO₂ = 1:0~0:1) |
| ZnO/Fe₂O₃ | 2~20 wt. % | K₂O/CeO₂ | 0-5 wt. % |
| (ZnO:Fe₂O₃ = | K₂CO₃/Li₂CO₃/Na₂CO₃ | (K₂O/CeO₂ = | MgO/MoO₃/Bi₂O₃ |
| 1:7~7:1) | (K : (Li + Na) = | 1:10~1:1) | (MgO:MoO₃ = 1:0~0:1; |
| | 0.01:1~1:0.01) | | MoO₃/Bi₂O₃ = 1:0~0:1) |
| Mixed Fe and/or Mn | 2~20 wt. % | K₂O/CeO₂ | 0-5 wt. % |
| containing oxides | K₂CO₃/Li₂CO₃/Na₂CO₃ | (K₂O/CeO₂ = | MgO/MoO₃/Bi₂O₃ |
| with a general | (K : (Li + Na) = | 1:10~1:1) | (MgO:MoO₃ = 1:0~0:1; |
| formula of CaₓA₁₋ₓ | 0.01:1~1:0.01) | | MoO₃/Bi₂O₃ = 1:0~0:1) |
| (Fe/Mn)_{y}B_{1-y}O₃, A = | | | |
| La, Sr, Ce, Ba, Pr, | | | |
| Sm, or Y; B = Co, Ti, | | | |
| Mg, Zr, Ce, Cr, or | | | |
| Mo. | | | |
| x = 0-1, y = 0-1* | | | |

*besides the ABO₃ type of oxide, mixed oxide with a Ruddlesden-Popper structure Aₙ₊₁BnO_{3n+1.}can also be used with a general formulation of (CaₓA₁₋ₓ)ₙ₊₁((Fe/Mn)yB_{1-y})nO₃ₙ₊₁

Redox Catalyst with COS component with SCH/COS +
DH in composite or core-shell forms

| SHC | | | |
|---|---|---|---|
| function/ | DH catalyst | | COS |
| component | component | DH dopant | component |
| CaO/La₂O₃/ | K₂O/Fe₂O₃ | 5 wt. % | K₂O/MnO₂ |
| MnO₂/CeO₂ | (K₂O/Fe₂O₃ = | MgO/ZrO₂/Bi₂O₃ | (K₂O/MnO₂ = |
| | 1:10~1:1) | (MgO:MoO₃ = 1:0.01~ | 1:10~10:1) |
| | | 0.01:1; | |
| | | MoO₃/Bi₂O₃ = 1:0.01~ | |
| | | 0.01:1) | |
| MgO/MnO₂/ | K₂O/Fe₂O₃ | 5 wt. % | Li₂O/MnO₂ |
| Fe₂O₃ | (K₂O/Fe₂O₃ = | MgO/CeO₂/Bi₂O₃ | (Li₂O/MnO₂ = |
| | 1:10~1:1) | (MgO:CeO₂ = 1:0.01~ | 1:10~10:1) |
| | | 0.01:1; | |
| | | CeO₂/Bi₂O₃ = 1:0.01~ | |
| | | 0.01:1) | |

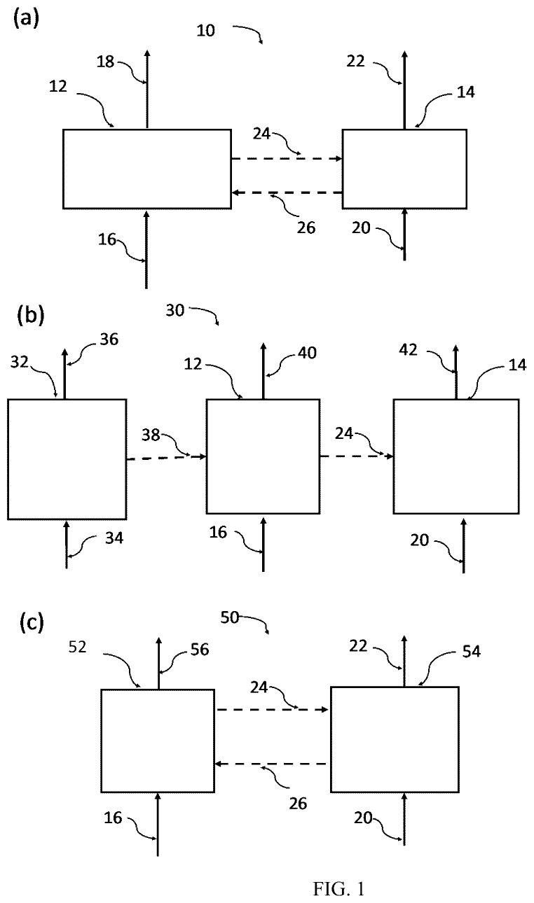

To minimize the COₓ selectivity of the redox-ODH from a process standpoint, H₂ generated from dehydrogenation could also be separated and collected and partially or entirely injected/recycled to the oxidized catalyst to: (a) partially remove the non-selective surface oxygen of the SHC component, as shown in FIG. 1(b); (b) the hydrogen can also react with surface carbonate species to convert the carbonate into CO which desorbs form the surface. In the dehydrogenation step, the H₂ could be partially converted by the SHC component. The remaining H₂ could be separated from other products and used to remove the nonselective surface oxygen species as well as surface carbonates prior to alkylaromatics injection.

Another novel approach to improve the catalyst performance and stability is also disclosed herein, denoted as Redox-induced Phase Transition ODH (R-PTODH), to oxidatively dehydrogenate alkylaromatics with the CO₂ resistance via solid-state reaction using a phase transition redox catalyst (PT-redox catalyst). The PT-redox catalyst comprises the COS component. As shown in FIG. 1(c), the proposed process can be separated into two steps: In the first step, catalyst may be activated by H₂, each time before contacting the alkylaromatics (as shown in FIG. 1(b)). Alkylaromatics are then catalytically converted to alkenylaromatics at a temperature range from 450° C. to 650° C., while partially or completely combusting H₂ with the PT-redox catalyst. The CO₂ co-generated could also be simultaneously captured by the PT-redox catalyst via chemical reaction, forming oxycarbonate or carbonate.

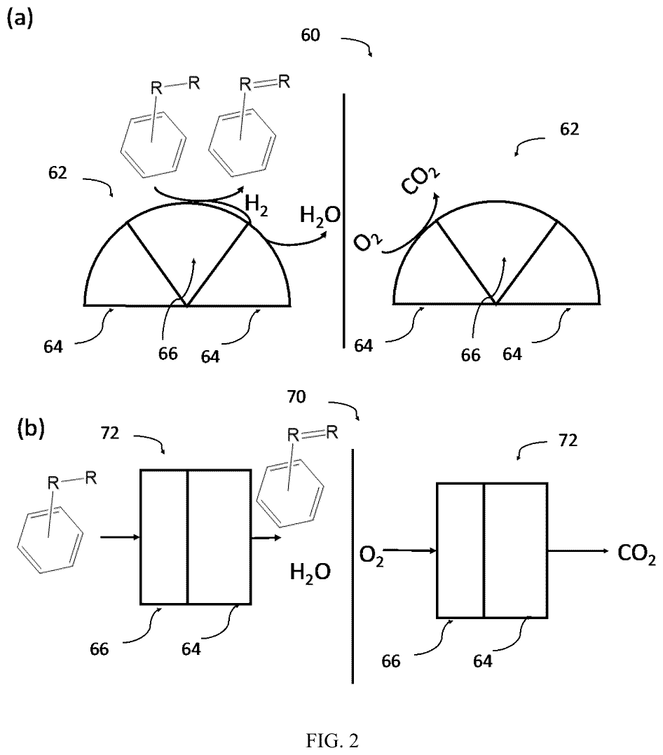

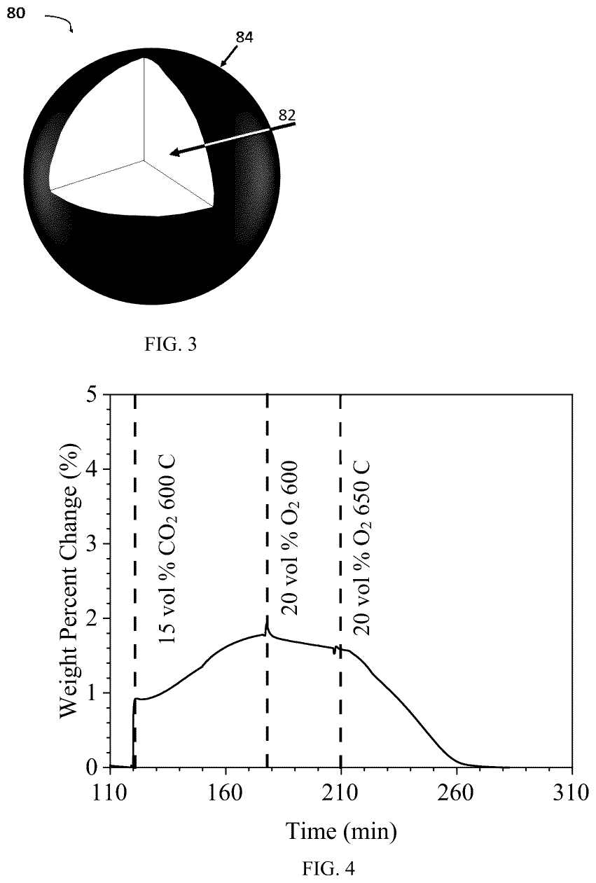

In the second step, the PT-redox catalyst is then regenerated in air, or other oxidizing gas such as steam or O₂ rich gas, which could require temperature swing from 650° C. to up to 850° C. The presence of oxidant would remove surface coke and provide lattice oxygen for the redox catalyst. The PT-redox catalyst would release the CO₂ from solid phase to gas phase, while regenerate the dehydrogenation component. This can result in a CO₂ rich and oxidant (such as O₂ or steam) lean gas stream. The PT-redox catalysts are designed to accommodate alkylaromatics catalytic conversion to alkenylaromatics and CO₂ resistance at the same time. This PT-redox catalyst is composed of three functional components: 1) the DH component to convert alkylaromatics to alkenylaromatics; 2) the oxygen storage phase which act as a SHC component; 3) CO₂ sorption component, capable of CO₂ absorption and desorption which facilitates CO₂ resistance of the catalysts. As shown in FIG. 2, these three functional components are combined into the same PT-redox catalyst on single or spatially adjacent active sites (at atomistic scales) or on spatially separated active sites. These functions could also be achieved separately and then physically integrated. The catalytic dehydrogenation, CO₂ capture and SHC could take place either sequentially or simultaneously in the temperature range of 350-900° C. FIG. 3 further illustrates a SHC redox catalyst design strategy where a mixed oxide core is covered with a potassium carbonate containing shell.

### EXAMPLES

### Comparative Example 1—K—Fe—O/Zn—Mn—O

1704.5 mg Zn(NO₃)₂ was wet-blended with 1506.1 mg Mn(NO₃)₂·4H₂O at 80° C. until dry. The resulting solids were calcined at 900° C. for 8 hours. The resulting solid was then wet-blended with 5602.9 mg Fe(NO₃)₃·9H₂O and 280.4 mg KNO₃ at 80° C. until dry. The resulting solid was calcined at 650° C. for 4 hours. 500 mg of the resulting solid were used as the catalyst bed. 0.022 mmol/min of 1,3 diisopropylbenzene (m-DIPB) was injected to the catalyst bed at 575° C. for 10 minutes, which resulted in m-DIPB conversion of 97.1%, m-DIPEB selectivity of 49.4% and COₓ selectivity of 8.8%, and coke selectivity of 41.8%.

Example 1 illustrates a redox catalyst with a dehydrogenation component (DH component) of K—Fe—O, a selective hydrogenation combustion component (SHC component) of a mixed zinc-manganese oxide in a core-shell configuration with the SHC component as the core and the DH component as the shell. Example 1 has no COₓ management (mitigation and resistance) component, resulting in m-DIPEB selectivity of 49.4% and COₓ selectivity of 8.8%, and coke selectivity of 41.8%.

### Example 2—K—Fe—O/(La_{0.8}Ca_{0.2})_{1.1}MnO_{3+δ}

162.8 mg La(NO₃)₃·6H₂O, 22.2 mg Ca(NO₃)₂·4H₂O and 107.3 mg Mn(NO₃)₂·4H₂O, 431.1 mg citric acid and 208.9 mg ethylene glycol were wet-blended at 80° C. until a uniform gel was formed. The gel was calcined at 900° C. for 8 hours. The resulting solid was wet-blended with 318 mg Fe(NO₃)₃·9H₂O and 79 mg KNO₃ at 80° C. until dry. The resulting solid was calcined at 650° C. for 4 hours. 500 mg of the calcined solid was used as the catalyst bed. 0.022 mmol/min of m-DIPB was injected to the catalyst bed at 575° C. for 10 minutes, which resulted in m-DIPB conversion of 95.3%, m-DIPEB selectivity of 78.4%, m-IPEC selectivity of 13.9%, COₓ selectivity of 2.4% and coke selectivity of 5.3%.

Example 2 illustrates a redox catalyst with a dehydrogenation component (DH) of K—Fe—O, an enhanced selective hydrogenation combustion function (enhanced SHC function) of a perovskite oxide of the formula La_{0.88}Ca_{0.22}MnO₃, representing ABO₃, with the A site partially substituted with the perovskite site component, Ca. The enhanced SHC function also serves as the COₓ management (mitigation and resistance) component. The redox catalyst was made in a core-shell configuration with the enhanced SHC function as the core and the DH component as the shell for each particle. Example 2 shows a reduction of COₓ selectivity to 2.4% and coke selectivity to 5.3% versus Comparative Example 1, with a COₓ selectivity of 8.8%, and coke selectivity of 41.8%.

### Example 3—K—Fe—O/(La_{0.8}Ca_{0.2})_{0.9}MnO_{3+δ}

7.417 g La(NO₃)₃·6H₂O, 1.011 g Ca(NO₃)₂·4H₂O, 5.972 Mn(NO₃)₂·4H₂O, 21.712 g citric acid, and 10.522 g ethylene glycol were wet-blended at 80° C. until a uniform gel was formed. The gel was calcined at 900° C. for 8 hours. The resulting solid was wet-blended with 15.912 g Fe(NO₃)₃·9H₂O and 3.982 g KNO₃ at 80° C. until dry. The resulting solid was calcined at 650° C. for 4 hours. 500 mg of the calcined solid were used as the catalyst bed. 0.022 mmol/min of m-DIPB was injected to the catalyst bed at 575° C. for 10 minutes, which resulted in DIPB conversion of 92.2%, m-DIPEB selectivity of 64.4%, m-IPEC selectivity of 29.0%, COₓ selectivity of 2.7%, and coke selectivity of 3.9%.

Example 3 illustrates a redox catalyst with a dehydrogenation component (DH) of K—Fe—O, COₓ management component consisting of an enhanced selective hydrogenation combustion function (enhanced SHC function) of a perovskite oxide of the formula La_{0.72}Ca_{0.18}MnO₃, representing ABO₃ with the perovskite site component being Ca. The redox catalyst was made in a core-shell configuration with the enhanced SHC function as the core and the DH component as the shell.

### Example 4—K—Fe—O/Ca—Fe—O

19.695 g Fe(NO₃)₃·9H₂O, 0.833 g of Ce(NH₄)₂(NO₃)₆, 0.1525 g of H₃₂Mo₇N₆O₂₈, 2.151 g KNO₃, 6.49 g Ca(NO₃)₂·4H₂O were blended with 48.910 g of citric acid and 31.587 g of ethylene glycol. The resulting solid was calcined at 850° C. for 4 hours. 3 g of the calcined solid was used as the catalyst bed. 0.088 mmol/min of 1,3-diisopropylbenzene (m-DIPB) was injected to the catalyst bed at 575° C. for 10 minutes, which resulted in m-DIPB conversion of 95.1%, m-DIPEB selectivity of 59.7%, m-IPEC selectivity of 28.4%, COₓ selectivity of 2.2% and, coke selectivity of 9.7%.

Example 4 illustrates a redox catalyst with a dehydrogenation component (DH component) of K—Fe—O, a selective hydrogenation combustion component (SHC component) of a mixed calcium iron-containing oxide. The catalyst also comprises a COₓ management component, as the DH component comprises a dopant which comprises the cations of cerium and molybdenum. The redox catalyst configuration from the direct mixing is a random disbursement of the DH component and the SHC component in each particle.

### Example 5—K—Fe—O/La_{0.7}Ca_{0.3}FeO₃

7.111 g La(NO₃)₃·6H₂O, 1.662 g Ca(NO₃)₂·4H₂O and 9.479 Fe(NO₃)₉·9H₂O, 22.54 g citric acid, and 10.92 g ethylene glycol were wet-blended at 80° C. until a uniform gel was formed. The gel was calcined at 900° C. for 8 hours. The calcined solid was wet-blended with 15.912 g Fe(NO₃)₃·9H₂O and 3.982 g KNO₃ at 80° C. until dry. The resulting solid was calcined at 650° C. for 4 hours. 500 mg of the calcined solid was used as the catalyst bed. 0.22 mmol/min of 1,3-diisopropylbenzene (m-DIPB) was injected to the catalyst bed at 575° C. for 10 minutes, which resulted in DIPB conversion of 96.5%, m-DIPEB selectivity of 56.9%, m-IPEC selectivity of 30.7%, COₓ selectivity of 5.2% and coke selectivity of 7.2%.

Example 5 illustrates a redox catalyst with a dehydrogenation component (DH) of K—Fe—O, COₓ management component consisting of an enhanced selective hydrogen combustion function (enhanced SHC function) of a perovskite oxide of the formula K—Fe—O/La_{0.7}Ca_{0.3}FeO₃, representing ABO₃ with the perovskite site component being Ca. The redox catalyst was made in a core-shell configuration with the enhanced SHC function as the core and the DH component as the shell.

### Example 6—K—Fe—O/LaPrO₃

6.605 g La(NO₃)₃·6H₂O and 6.635 Pr(NO₃)₃·6H₂O, 14.65 g citric acid and 7.10 g ethylene glycol were wet-blended at 80° C. until a uniform gel was formed. The gel was calcined at 900° C. for 8 hours. The calcined solid was then wet-blended with 22.63 g Fe(NO₃)₃·9H₂O and 1.13 g KNO₃ at 80° C. until dry. The resulting solid was calcined at 650° C. for 4 hours. 500 mg of the calcined solid was used as the catalyst bed. 0.22 mmol/min of m-DIPB was injected to the catalyst bed at 575° C. for 10 minutes, which resulted in DIPB conversion of 96.9%, m-DIPEB selectivity of 51.2%, m-IPEC selectivity of 6.3%, COₓ selectivity of 4.5% and coke selectivity of 38.1%.

Example 6 illustrates a redox catalyst with a dehydrogenation component (DH component) of K—Fe—O, a selective hydrogen combustion component (SHC component) of a perovskite oxide of the formula LaPrO₃. The redox catalyst was made in a core-shell configuration with the SHC component as the core and the DH component as the shell for each particle.

### Example 7-PT-RDH: Mn Doped-KFeO₂

3.015 g KNO₃, 7.531 mg Fe(NO₃)₃·9H₂O and 0.935 g Mn(NO₃)₂·4H₂O, were wet-blended at 80° C. until a uniform gel was formed. The resulting mixture was calcined at 900° C. for 8 hours. 10 mg of the resulting solid was used for the thermogravimetric analysis. 15 vol. % CO₂ (balance Ar) was flowed at 600° C. for 1 hour. Then 20 vol. % 02 (balance Ar) was flowed at 650° C. for 1 hour. The result, indicating that the mixture could release all CO₂ in oxidative condition at slightly elevated temperature, is shown in FIG. 4.

Example 7 illustrates a catalyst with a dehydrogenation component (DH component) of K—Fe—O, no selective hydrogen combustion component (SHC component), and a CO₂ sorption component of KMn⁴Oₓ. Example 7 shows how the CO₂ sorption component (COS component) absorbs CO₂ as well as how the CO₂ is liberated when exposed to an oxygen source, 20 vol. % O₂ (balance Ar).

Listed below are several advantages of the invention disclosed herein compared to the prior art and/or non-limiting aspects of the redox catalyst and its use in the oxidative dehydrogenation of alkylaromatics.
1) SHC dopants decreases the CO₂ selectivity and increase single pass yield of desired unsaturated aromatics by reducing unselective lattice oxygen at the surface via abovementioned possibilities.
2) DH material dopants increase the lifetime of the catalyst and avoid permanent CO₂ poisoning.
3) In the R-PTODH process (the RP-TODH catalyst comprises a COS component), the CO₂ is dynamically absorbed in the dehydrogenation step, and released in the regeneration step, therefore eliminating the DH component deactivation.
4) The CO₂ absorbed during the dehydrogenation step results in a CO₂-free stream, removing the requirement for a CO₂ capture unit and preventing carbonation reactions downstream. The CO₂ release at the regeneration step could result in a highly concentrated CO₂ stream, facilitating easy CO₂ capture and storage.
5) The SHC would remove H₂ from dehydrogenation process timely, so that the thermodynamic equilibrium limitation is largely removed, leading to higher single pass conversion and yield.
6) The detailed design of the SHC materials would inherently decrease the COₓ selectivity.
7) The process does not require co-feeding gaseous oxygen with the alkyl aromatic feedstock. Usage of externally added steam can also be reduced or eliminated.
8) The injection of H₂, prior to the dehydrogenation step, ranging from 1 vol. % to 100 vol. % could suppress the CO₂ selectivity hydrocarbons resulting in a higher selectivity by consumption of the nonselective oxygen in the redox catalyst.
9) The supply of oxygen from the reduction of the redox catalyst may make the net reaction endothermic, heat neutral, or even exothermic. While the regeneration of reduced redox catalyst may supplement the heat for the R-ODH process.
10) Alkyl aromatic hydrocarbon is used as the lift gas in the R-ODH reactor, contacting the high temperature, highly oxidized redox catalyst so that
   a. C—C bond in the alkyl aromatic hydrocarbon is converted to C═C bond in the alkene aromatic hydrocarbon followed by the selective hydrogen combustion to water;
   b. In dehydrogenation step, two or more hydrogen from α-carbon will be abstracted by the dehydrogenation component sequentially and/or simultaneously;
   c. The steam produced from selective hydrogen combustion in dehydrogenation step may suppress the formation of coke on the reactor and catalyst particles
   d. The CO₂ produced from full oxidation of alkylaromatics may be absorbed by the COS component.
11) An inert, inexpensive heat transport carrier such as α-Al₂O₃ of silicon carbide can be cycled with these oxygen carrying redox catalysts so that thermal sufficiency of the reactor can be maintained with
   a. Lower charges of redox catalyst particles, and/or
   b. Lower overall circulation rate
12) The redox catalysts contain both catalytic dehydrogenation and oxygen storage components. They conduct R-ODH at high temperature (400-700° C.). In the reduction step, C—H bond in the alkane side chain attached to benzene ring is catalytically dehydrogenated while the hydrogen produced from this dehydrogenation is selectively combusted to water by redox catalyst. To prevent over-reduction of the redox catalysts and ensure the conversion of hydrogen from dehydrogenation, which may cause coke formation and catalyst deactivation, a suitable gas-solid contact time is maintained (0.1-120 s). In the oxidation step for the regeneration of redox catalyst, a full oxidation is conducted to replenish the redox catalyst. In some case, H₂ injection before the dehydrogenation step may reduce CO₂ selectivity and remove surface carbonates. To prevent over-reduction, a suitable concentration and contact time can be used, (1˜10 vol. % and 0.1 s˜60 s). In some cases, the regeneration degree of redox catalyst may be controlled by partial regeneration, oxidation with dilute oxygen, use of soft oxidants (e.g., CO₂ or H₂O), and/or pulse of the oxidants to avoid over-reoxidation and non-selective oxygen formation in redox catalyst. In the cyclic redox of R-ODH or R-PTODH, the reduction and oxidation steps are alternately conducted for the efficient dehydrogenation of alkyl aromatic hydrocarbons as well as hydrogen selective combustion. Many reactor configurations including fluid bed, fixed bed, moving bed, simulated moving bed, or rotating bed can be suitable for R-ODH.
   a. In another embodiment, the redox catalyst should possess the functions of both catalytic dehydrogenation and hydrogen selective combustion. The redox catalyst may consist of catalytic dehydrogenation component, oxygen storage component, and/or CO₂ resistant dopants, and/or phase transition components. In addition, both functions may be combined together or directly mixed by the three components when being used as the redox catalyst. For some redox catalyst, all functions may be integrated in one component or metal oxide.
   b. In the regeneration step, oxidants including air, oxygen, water, CO₂, and their mixture may be used to replenish the lattice oxygen in redox catalysts, CO₂ may be released in this step.
   c. In dehydrogenation step, one or more C—C bonds may be selectively converted into double or triple bonds by using specifically-designed redox catalysts, CO₂ may be absorbed by the catalyst.
   d. Heat management in the R-ODH step may be regulated by the redox catalyst as discussed earlier for improved heat integration and/or simplified reactor design.
13) Using the redox catalysts and R-ODH scheme described herein, in some aspects, the redox catalyst is packed in two alternately recycled fixed-bed reactors as shown in FIG. 1(a) or 1(b) or 1(c). The inlet and outlet gas lines are managed by a 4-way valve to ensure continuously input of alkyl aromatic hydrocarbons and oxidant as well as the output of dehydrogenation products. The system can be further configured to include multiple packed bed reactors in parallel. Each reactor can be further configured to include multiple layers of catalysts with varying compositions and functionality.
14) The potential alkylbenzene feedstocks to be dehydrogenated include ethylbenzene, di-ethylbenzene, diisopropylbenzene, cumene, among others.
15) redox catalyst comprising a CO₂ sorption component, can absorb CO₂ cogenerated when feeding alkylaromatics, and desorb CO₂ when feeding oxidant to dynamically preserve the catalytic dehydrogenation component.
16) Using an oxygen storage material that enables the release of oxygen at low temperatures in the redox catalyst may achieve an autothermal thermal dehydrogenation in the reduction step.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

1. A redox catalyst comprising: a catalytic dehydration (DH) component, a COₓ management (mitigation and resistance) component, and an optional enhanced selective hydrogen combustion (SHC) function within the COₓ management component or an optional SHC component,
wherein the redox catalyst comprises one of the optional enhanced SHC function or the optional SHC component,
wherein the DH component comprises a first metal (iron and/or cerium)-containing oxide,
wherein the COₓ management component is selected from the group consisting of
i. a first dopant, wherein the DH component comprises the first dopant, and the first dopant comprises a cation of a second metal;
ii. a CO₂ sorption (COS) component, wherein the COS component comprises an oxide and/or hydroxide of a first mixed-metal, wherein the oxide and/or hydroxide of the first mixed-metal is capable of reacting with CO₂ to form the corresponding oxycarbonate and/or carbonate; and/or
iii. the optional enhanced SHC function comprising a second mixed-metal oxide comprising reactive lattice oxygen, and
a. a perovskite site component, wherein the second mixed-metal oxide comprises the perovskite site component;
b. a selectivity enhancing shell on the outside of the enhanced SHC function, wherein the shell comprises oxides, carbonates, sulfates, phosphates, pyrophosphates, molybdates, tungstates, nitrates, nitride, chloride, bromide, and/or iodide of a third metal; and/or
c. a second dopant, wherein the enhanced SHC function comprises the second dopant, wherein the second dopant comprises a carbonate and/or oxide of a fourth metal,
wherein the optional SHC component comprises the second mixed-metal oxide, and
wherein the first metal, the second metal, third metal and/or the fourth metal are independently selected and can be the same or different.

2. The redox catalyst of claim 1,
wherein the first metal is selected from the group consisting of alkali metals,
wherein the second metal is selected from the group consisting of alkali metals and/or alkaline earth metals, transition metals, post-transition metals, and/or lanthanide metals,
wherein the oxide and/or the hydroxide of the first mixed-metal is selected from the group consisting of alkali metals, alkaline earth metals, and/or transition metals,
wherein the second mixed-metal is selected from the group consisting of alkaline earth metals, transition metals, and/or lanthanide metals,
wherein the perovskite site component is selected from the group consisting of alkaline earth metal and/or lanthanide metals,
wherein the third metal is selected from the group consisting of alkali metals, alkaline earth metals, and/or rare earth metals, and
wherein the fourth metal is selected from the group consisting of alkali metals and/or alkali earth metals.

3. The redox catalyst of claim 1, wherein a configuration of the redox catalyst is selected from the group consisting of a single particle comprising the DH component, the optional enhanced SHC function, the optional SHC component and/or the COS component; multiple particles wherein the DH component is in a distinct particle from the optional enhanced SHC function, the optional SHC component, and/or COS component;
and/or multiple particles wherein each particle independently comprises the DH component, the optional enhanced SHC function, the optional the SHC component, and/or the COS component.

4. The redox catalyst of claim 1, wherein the DH component comprises the first metal (iron and/or cerium)-containing oxide, and the first metal (iron and/or cerium)-containing oxide is selected from the group consisting of
i. a potassium iron-containing oxide selected from the group consisting of K—Fe—O, K₂O/MeFe₂O₄, and/or K₂O/Ca₂Fe₂O₅, or a potassium iron-containing-oxide comprising K—Fe—O, mixed K-Mₓ oxides, mixed Fe-Mₓ oxides, and/or mixed K—Fe-Mₓ oxides, wherein Mₓ is selected from the group consisting of Ca, Mo, Mn, and/or Cr;
ii. a lithium iron-containing oxide selected from the group consisting of Li—Fe—O, Li₂O/MeFe₂O₄, and/or Li₂O/Ca₂Fe₂O₅;
iii. a sodium iron-containing oxide selected from the group consisting of Na—Fe—O, Na₂O/MeFe₂O₄, and/or Na₂O/Ca₂Fe₂O₅;
iv. a zinc iron-containing oxide selected from the group consisting of Zn—Fe—O, Zn₂O/MeFe₂O₄, and/or Zn₂O/Ca₂Fe₂O₅; and/or
v. a cerium-containing-oxide selected from the group consisting of K—Ce—O, Li₂O/CeO₂, Na₂O/CeO₂, and/or Zn₂O/CeO₂,
wherein Me is independently selected from the group consisting of Mn, Cu, Co, Zn, and/or Ni.

5. The redox catalyst of claim 1, wherein the first metal (iron and/or cerium)-containing oxide comprises a potassium iron-containing oxide, and wherein a weight ratio of K₂O to Fe₂O₃ ranges from 1:10 to 1:1, or from 1:10 to 1:2, or from 1:10 to 1:4, or from 1:10 to 1:6, or from 1:10 to 1:8, or from 1:8 to 1:1, or from 1:8 to 1:2, or from 1:8 to 1:4, or from 1:8 to 1:6, or from 1:6 to 1:1, or from 1:6 to 1:2, or from 1:6 to 1:4, or from 1:4 to 1:1 or from 1:4 to 1:2, or from 1:2 to 1:1; and/or wherein the first metal (iron and/or cerium)-containing oxide comprises a potassium cerium-containing oxide, and wherein a weight ratio of K₂O to CeO₂ ranges from 1:10 to 1:1, or from 1:10 to 1:2, or from 1:10 to 1:4, or from 1:10 to 1:6, or from 1:10 to 1:8, or from 1:8 to 1:1, or from 1:8 to 1:2, or from 1:8 to 1:4, or from 1:8 to 1:6, or from 1:6 to 1:1, or from 1:6 to 1:2, or from 1:6 to 1:4, or from 1:4 to 1:1 or from 1:4 to 1:2, or from 1:2 to 1:1, or from 1:6 to 2:1, or from 1:4 to 2:1, or from 1:2 to 2:1 or from 1:1 to 2:1.

6. The redox catalyst of claim 1, wherein the DH component comprises the first dopant and the second metal is selected from the group consisting of Ag, Bi, Ca, Ce, Cr, Li, Ga, Mg, Mo, Mn, Ti, V and/or Zr, and wherein an ionic compound comprising the cation of the second metal is selected from the group consisting of oxides, carbonates, sulfates, phosphates, pyrophosphates, molybdates, tungstates, and/or nitrates; or wherein the DH component comprises the first dopant and the second metal is selected from the group consisting of Ag, Bi, Ce, Cr, Ga, and/or Mo, and wherein an ionic compound comprising the cation of the second metal is selected from the group consisting of oxides, carbonates, sulfates, phosphates, pyrophosphates, molybdates, tungstates, and/or nitrates.

7. The redox catalyst of claim 1, wherein the second mixed-metal oxide is selected from the group consisting of
i. an iron-containing oxide, wherein the iron-containing oxide optionally comprises an alkaline earth metal and/or a transition metal other than iron;
ii. a vanadium oxide;
iii. bismuth molybdate or molybdenum oxide;
iv. an ionic compound comprising the cation of Ti and an ion selected from the group consisting of MgO/MoO₃/AgO; MgO/Cr₂O₃/AgO; MgO/Cr₂O₃/CeO₂; MgO/MoO₃/ZrO₂; MgO/MoO₃/Bi₂O₃; MgO/ZrO₂/Bi₂O₃; and/or MgO/CeO₂/Bi₂O₃; and/or
v. perovskite oxides or oxides selected from the group consisting of ABO₃, A₂B₂O₅, and/or Aₙ₊₁BₙO₃ₙ₊₁, wherein A is selected from the group consisting of alkaline earth metals and/or lanthanide metals, and B is selected from the group consisting of transition metals and/or lanthanide metals, and wherein n is a positive integer or wherein n is 1, 2, 3, or 4.

8. The redox catalyst of claim 1, wherein the redox catalyst comprises the optional enhanced SHC function comprising the second mixed-metal oxide and the selectivity enhancing shell, wherein the third metal is selected from the group consisting of Li, Na, K, Rb, Cs, Be, Mg, Ca, Sr, and/or Ba; or wherein the third metal is selected from the group consisting of Li, Na, and/or K.

9. The redox catalyst of claim 1, wherein the COₓ management component comprises the COS component comprising the oxide and/or the hydroxide of the first mixed-metal, and wherein the first mixed-metal is selected from the group consisting of Li, Na, K, V, Mn, Fe, Co, and/or Cu; or wherein the COₓ management component comprises the COS component comprising the oxide and/or hydroxide of the first mixed-metal, and wherein the first mixed-metal oxide or hydroxide comprises K and a metal selected from the group consisting of Li, Na, V, Mn, Fe, Co, and/or Cu.

10. The redox catalyst of claim 1, wherein a molar ratio of the first metal (iron and/or cerium)-containing oxide to the second mixed-metal oxide ranges from 1:10 to 10:1, or from 1:5 to 5:1.

11. A process for producing an olefinic compound, comprising:
(a) providing a redox catalyst of claim 1 in a reactor;
(b) optionally an oxidized redox catalyst pre-reduction step comprising introducing hydrogen into the reactor to produce steam and a pre-reduced SHC component, followed by stopping introducing the hydrogen;
(c) a redox catalyst dehydrogenation and selective hydrogen combustion step comprising introducing a dehydrogenation reactant into the reactor,
(i) wherein the dehydrogenation reactant contacts the DH component to produce the olefinic compound and hydrogen, and
(ii) wherein the hydrogen contacts the optional enhanced SHC function or the optional SHC component to produce steam and a reduced second mixed-metal oxide, wherein at least 10% of the hydrogen released in step c(i) is converted to steam;
(d) removing the olefinic compound and the steam from the reactor;
(e) stopping introducing the dehydrogenation reactant;
(f) a redox catalyst regeneration step comprising introducing an oxygen source into the reactor, wherein the reduced second mixed-metal oxide is re-oxidized to the second mixed-metal oxide;
(g) stopping introducing the oxygen source; and
(h) a cycle repeating step comprising repeating steps (b) through (g) at least once.

12. The process of claim 11, wherein the dehydrogenation reactant comprises an alkyl aromatic hydrocarbon or a substituted alkyl aromatic hydrocarbon and the olefinic compound comprises an alkene aromatic hydrocarbon or substituted alkene aromatic hydrocarbon, respectively; or wherein the dehydrogenation reactant comprises alkylated benzenes, alkylated thiophenes, alkylated furans, oligomers of alkylated benzenes, oligomers of alkylated thiophenes, and/or oligomers of alkylated furans; or wherein the dehydrogenation reactant comprises ethylbenzene, di-ethylbenzene, diisopropylbenzene, and/or cumene; or wherein the dehydrogenation reactant comprises 1,2-diisopropylbenzene, 1,3-diisopropylbenzene and/or 1,4-diisopropeylbenzene, and the olefinic compound comprises 1,2-diisopropenylbenzene, 1,3-diisopropenylbenzene and/or 1,4-diisopropenylbenzene.

13. The process of claim 11, wherein a reactor temperature during the redox catalyst dehydrogenation and selective hydrogen combustion step (c) ranges from 350° C. to 900° C., or from 400° C. to 700° C., or from 450° C. to 650° C.

14. The process of claim 11, wherein the dehydrogenation reactant contacts the redox catalyst for a contact time ranging from 0.1 s to 2,000 s, or from 0.1 s to 1,000 s, or from 0.1 s to 500 s, or from 0.1 sec to 250 s, or from 0.1 s to 120 s, or from 0.1 s to 60 s, or from 10 s to 2,000 s, or from 10 s to 1,000 s, or from 10 s to 500 s, or from 10 sec to 250 s, or from 10 s to 120 s, or from 10 s to 60 s, or from 30 s to 2,000 s, or from 30 s to 1,000 s, or from 30 s to 500 s, or from 30 s to 250 s, or from 30 s to 120 s, or from 30 s to 60 s.

15. The process of claim 11, wherein the redox catalyst dehydrogenation and selective hydrogen combustion step (c) occurs for a time range of 5 minutes to 30 minutes or a time range of 5 minutes to 2 hours, and wherein at least 15%, or at least 20%, or at least 25%, or at least 30%, or at least 40%, or at least 50% of the hydrogen released in step c(i) is converted to steam.

16. The process of claim 11, and wherein the redox catalyst regeneration step (f) occurs for a time range of 5 minutes to 30 minutes, or 5 minutes to 2 hours.

17. The process of claim 11, wherein the oxidized redox catalyst pre-reduction step (b) occurs and wherein a gas stream is introduced to the reactor, wherein the gas stream comprises between 1 vol. % to 10 vol. % hydrogen and 99 vol. % to 90 vol. % inerts; and wherein the gas stream contacts the redox catalyst for a contact time ranging from 0.1 s to 2,000 s, or from 0.1 s to 1,000 s, or from 0.1 s to 500 s, or from 0.1 sec to 250 s, or from 0.1 s to 120 s, 0.1 s to 60 s, or from 0.1 sec to 45 s, or from 0.1 sec to 30 sec, or from 10 s to 2,000 s, or from 10 s to 1,000 s, or from 10 s to 500 s, or from 10 sec to 250 s, or from 10 s to 120 s, or from 10 s to 60 s, or from 10 s to 45 s, or from 10 sec to 30 sec, and wherein the oxidized redox catalyst prereduction step (b) occurs for a time range of 5 minutes to 30 minutes.

18. The process of claim 11, wherein when the cycle repeating step (h) has occurred between 10 and 1,000 time, or between 10 and 500 times, or between 10 and 250 times, or between 10 and 100 times, or between 50 and 1,000 times, or between 50 and 500 times, or between 50 and 250 times, or between 50 and 100 times; and wherein the oxygen source introduced in the next redox catalyst regeneration step (f) comprises greater than 50 vol. % O₂, or greater than 60 vol. % O₂, or greater than 70 vol. % O₂, or greater than 80 vol. % O₂.

19. The process of claim 11, wherein the reactor is operated in a configuration during steps (b) through (g) selected from the group consisting of fluid bed, fixed bed, moving bed, simulated moving bed, or rotating bed, and wherein an operating pressure ranges from 0.1 bar to 5 bar.

20. The process of claim 12, wherein the dehydrogenation reactant into the reactor in step (c) occurs in a feed stream comprising the dehydrogenation reactant, steam, and inert; wherein the mole percent of dehydrogenation reactant in the feed ranges from 1 mole % to 40 mole %, or from 1 mole % to 20 mole %, or 1 mole % to 10 mole %; or from 3 mole % to 40 mole %, or from 3 mole % to 20 mole %, or 3 mole % to 10 mole %; wherein the mole percent of steam in the feed stream ranges from 1 mole % to 60 mole %, or from 1 mole % to 40 mole % or from 1 mole % to 20 mole %, or 5 mole % to 60 mole %, or from 5 mole % to 40 mole % or from 5 mole % to 20 mole %, or 10 mole % to 60 mole %, or from 10 mole % to 40 mole % or from 10 mole % to 20 mole %; wherein the mole percent of inert in the feed stream ranges from 20 mole % to 90 mole %, or from 20 mole % to 80 mole %, or 20 mole % to 60 mole %; and wherein the inert comprises nitrogen and/or argon.
